(19) United States
(12) Patent Application Publication
(10) Pub. No.: US 2020/0305966 A1
(43) Pub. Date: Oct. 1, 2020

(54) DEVICE AND METHOD FOR TREATING TISSUE

(71) Applicant: Candela Corporation, Wayland, MA (US)

(72) Inventor: Dany Berube, Milpitas, CA (US)

(73) Assignee: Candela Corporation

(21) Appl. No.: 16/367,844

(22) Filed: Mar. 28, 2019

(51) Int. Cl. A61B 18/14; A61B 18/12

(52) U.S. Cl. CPC A61B 18/1477; A61B 2018/00791; A61B 18/1206

(57) ABSTRACT

A device and method for treating tissue. Two or more pairs of needle electrodes are spaced about a zone and each pair of needle electrodes includes a first needle electrode on one side of the zone and a second needle electrode on an opposite side of the zone. An energy source for each pair of needle electrodes is configured to induce current from the first needle electrode of the pair to only the second needle electrode of the pair through the interior of the zone to increase the total current density and temperature in the interior of the zone.

### FIELD OF THE INVENTION

The subject invention relates to bipolar RF needle electrode treatment devices and systems.

### BACKGROUND OF THE INVENTION

Bipolar RF needle electrodes have been used for various skin treatments. See U.S. Pat. Nos. 9,095,357 and 9,744,371 both incorporated herein by this reference. In some cases, an array of closely spaced needles are inserted into the skin and energized causing current to flow between the needles creating thermal damage to the tissue. Skin tightening, reduction of wrinkles, and cellulite reduction are common treatments. See also U.S. Pat. No. 8,845,630 and published U.S. Patent Application No. 2012/0143178 both incorporated herein in by this reference.

### SUMMARY OF THE INVENTION

For some treatments, however, it would be beneficial to increase the volume of the thermal injuries. Examples include cellulite reduction, tumor ablation, abnormal tissue growth treatment, for example treatment of uterine fibroid tissue, and/or other treatments. For cellulite reductions, the subcutaneous fat to be treated includes two layers separated by superficial fascia. For women with cellulite, the inner layer thickness was found to be about five fold thicker than for women without cellulite (23 mm v. 4 mm). The total subcutis thickness for cellulite patients was between 2.7 and 50 cm. A higher percentage of fibrous septae perpendicular to skin surface was also noted in cellulite patients. See Rawlings A. V., Cellulite and its Treatment, International Journal of Cosmetic Science, 2006, 28. 175-190 incorporated herein by this reference.

Featured is a tissue treatment device which increases the total current density in the interior of a larger volume treatment zone thereby improving the uniformity of the temperature profile. In one particular example, the tissue treatment device, when used in conjunction with cellulite treatment, or fat removal procedure, creates thermal injuries in the entire or a significant portion of the subcutis space by increasing the volume of thermal injury.

One preferred device comprises two or more pairs of needle electrodes spaced about a zone wherein each pair of needle electrodes includes a first needle electrode on one side of the zone and a second needle electrode on an opposite side of the zone. There is an energy source for each pair of needle electrodes configured to induce current from the first needle electrode of the pair to only the second needle electrode of the pair in a cross firing manner through the interior of the zone to increase the total current density and temperature in the interior of the zone.

Each pair of needle electrodes preferably defines a plane intersecting the center of the zone and the planes defined by each pair of needle electrodes preferably intersect each other at the center of the zone. In another embodiment, the planes defined by each pair of needle electrodes intersect away from the center of the zone.

In one version, the energy source for each pair of needle electrodes is an RF generator connected to the first and second needle electrodes of each pair. The RF generators of each pair of needle electrodes are preferably electrically isolated from the RF generators of all other pairs of needle electrodes.

In some embodiments, the needle electrodes are all equidistantly spaced from the center of the zone. The device may further include a cartridge carrying the pairs of needle electrodes and an applicator for receiving the cartridge. Preferably, the cartridge is removeable from and insertable into the applicator.

In some embodiments, each needle electrode has an active length of between 0.5 and 40 mm, the needle electrodes of each pair of needle electrodes are spaced apart from each other by a distance of between 1.0 and 10 mm, and each needle electrode is spaced apart from each adjacent needle electrode by a distance of between 0.71 and 7.1 mm.

One or more needle electrodes may further include a temperature sensor. The device controller for the energy sources may be responsive to the one or more temperature sensors. In one example, the controller is configured to control the energy sources based on an output signal of the one or more temperature sensors. The device controller may be configured to automatically adjust the temperature in the zone to between 40° C. and 48° C. from between 30 seconds and 30 minutes.

Also featured is a method of treating tissue wherein two or more pairs of needle electrodes are inserted into a zone of tissue. Each pair of needle electrodes includes a first needle on one side of the zone and a second needle on an opposite side of the zone. The method includes electrically isolating each pair of needle electrodes from all other pairs of needle electrodes and inducing current from the first needle of each pair to only the second needle electrode of the pair through the interior of the zone increasing the total current density and temperature at the interior of the zone.

Also featured is a device for treating tissue including pairs of needle electrodes electrically isolated from each other and spaced about a tissue zone having a central volume. Each pair of needle electrodes define a plane intersecting the central volume of the tissue zone. The planes defined by each pair of needle electrodes intersecting each other at the central volume of the tissue zone. The device further includes means for inducing a current from the first needle electrode of each pair to only the second needle electrode of each pair across the central volume of the tissue zone.

Also featured is a method of treating tissue including inserting a plurality of needles into a tissue treatment zone, spacing the needles from a central volume of the tissue treatment zone, inducing current from each needle on one side of the central volume of the treatment zone through the central volume of the treatment zone to only another needle on an opposite side of the central volume of the treatment zone, and controlling the induced currents to intersect in the central volume of the treatment zone increasing the energy deposition in the central volume of the treatment zone.

The subject invention, however, in other embodiments, need not achieve all these objectives and the claims hereof should not be limited to structures or methods capable of achieving these objectives.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a schematic view of a prior art bipolar RF needle electrode treatment device;

FIG. 2 shows a thermal profile around and between a pair of needle electrodes of the electrode needle array shown in FIG. 1;

FIG. 3 is a schematic view of a tissue treating device in accordance with an example of the invention;

FIG. 4 shows the temperature profile for the needle electrode arrangement of FIG. 3 and an increase in the temperature at the interior of the treatment volume;

FIGS. 5A and 5B are further views of the thermal profile of the needle electrode arrangement of FIG. 3;

FIG. 6 is a graph showing the temperature gradient for a calculated temperature smoothness ratio;

FIG. 7 is a graph showing the thermal profile for two pairs of needle electrodes spaced 7.5 mm apart;

FIG. 8 is a graph showing the thermal gradient between two pairs of needles at different treatment application time periods;

FIG. 9 is a graph of the temperature profile for two needle pairs when the calculated smoothness ratio was 1.52;

FIG. 10 is a schematic view showing an example of a cartridge and applicator unit in one particular example of the treatment device;

FIGS. 11A-B are views showing the temperature profile of a treatment volume when two treatment cells are deployed each including two needle pairs; and

FIG. 12 is a schematic view showing the temperature profile of a treatment volume when three electrode needle pairs are used.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

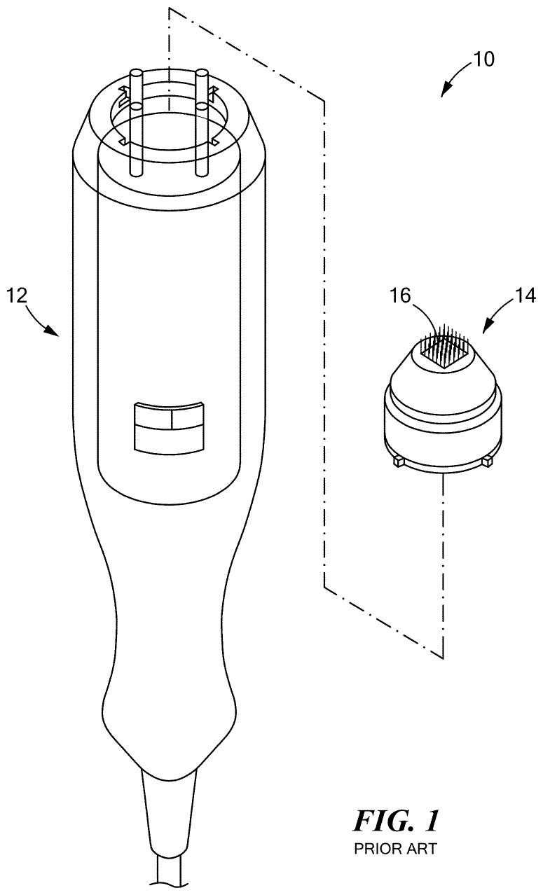

FIG. 1 shows a prior art RF needle electrode treatment apparatus including hand piece 12 and cartridge 14 with closely spaced micronedle electrodes 16 in an array. Typically, every other row of the needle electrodes is connected to positive terminal of an RF generator and the rows of needle electrodes in between are connected to the negative terminal of the RF generator.

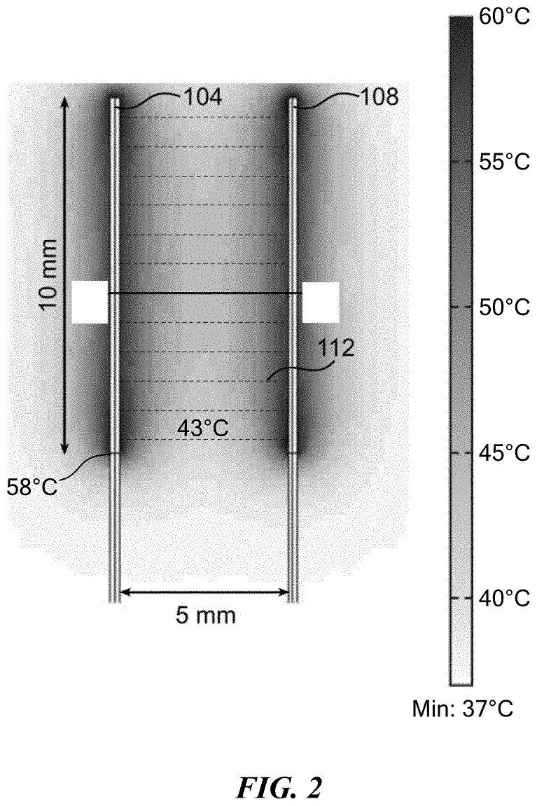

FIG. 2 shows the results of a Finite Element Analysis study and the thermal profile of a pair of energy delivering needle electrodes 104 and 108 spaced by a distance of 5 mm from each other and inserted in subcutaneous tissue (fat). A difference of potential of 60 volts was applied to the electrodes for 20 seconds. The needles had an active length of 10 mm being defined as the active needle part with direct metal-tissue contact Reference numeral 112 marks RF energy induced current streamlines between electrodes 104 and 108. It is clear that the temperature levels are higher in the vicinity of the needles (about 58° C.) than at the midpoint between the two needles where the temperature is only about 43° C. The result was a substantial 15° C. temperature difference between the maximum and minimum temperatures in the tissue volume.

This result shows the difficulty of creating uniform thermal profiles in subcutis (fat) where the two needles of a pair are spaced apart to a distance of about 5 mm or more in a larger volume of tissue to be treated. This is partly explained by considering the low thermal conductivity of subcutis which is a good thermal isolator. Thermal energy does not propagate well and can produce steep thermal gradients within the tissue.

The issue of creating fairly uniform thermal profiles in biological tissue (subcutis and others) with needle-type electrodes spaced about 5 mm apart can be further explained by considering the divergence of the current lines. When the two energy delivering needle electrodes of a pair are too far away from each other, the current density in the tissue to be treated located at the midpoint between the two energy delivering needle electrodes is very low compared to the current density in the vicinity of the electrodes creating a zone of weak energy deposition in the tissue. As a consequence, the power deposited at the midpoint between the electrodes is lower than in the immediate vicinity of the electrode and the temperature elevation is consequently much lower.

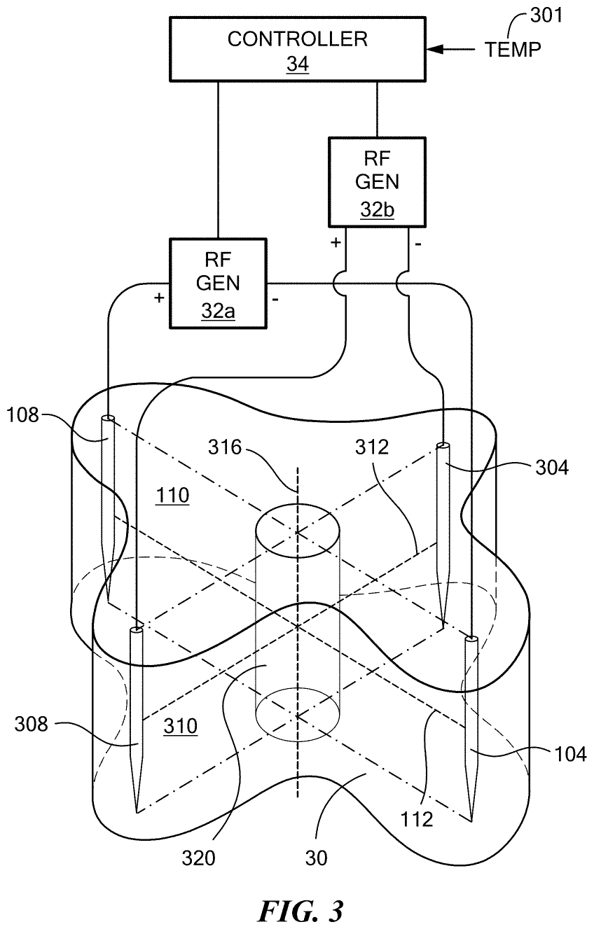

To increase the RF energy deposition in the middle section between the energy delivering needle electrodes 104 and 108, a second pair of RF energy delivering needle electrodes 304 and 308, FIG. 3 were introduced. Preferably, the second pair of electrodes 304, 308 are added in another spatial plane in which the electrical current lines would intersect in the vicinity of the central area 320 of a treatment volume 30 to increase the total current density in the central area 320 which thus increases the energy deposition in that volume and consequently increases the temperature elevation of the central area to increase the thermal profile uniformity. The two non-coplanar RF electrode needle pairs 104, 108 and 304, 308 work together to increase the energy deposition in the area 320 of weak energy deposition in tissue. In theory, the energy deposition in a zone or volume 320 of weak energy deposition would be doubled if two pairs of non-coplanar RF electrode needles 104, 108 and 304, 308 are used and where their zones of weak energy deposition coincide. In one example, the first plane 110 defined by a pair of RF electrodes 104 and 108 and second plane 310 defined by a pair of RF electrodes 304 and 308 intersect at line 316 defining the longitudinal axis of volume 320.

This concept could be further expanded by using more than two pairs of RF needle electrode pairs where their zones of weak energy deposition coincide so they can work together to create a uniform thermal profile within the volume defined by the active portions of the needles.

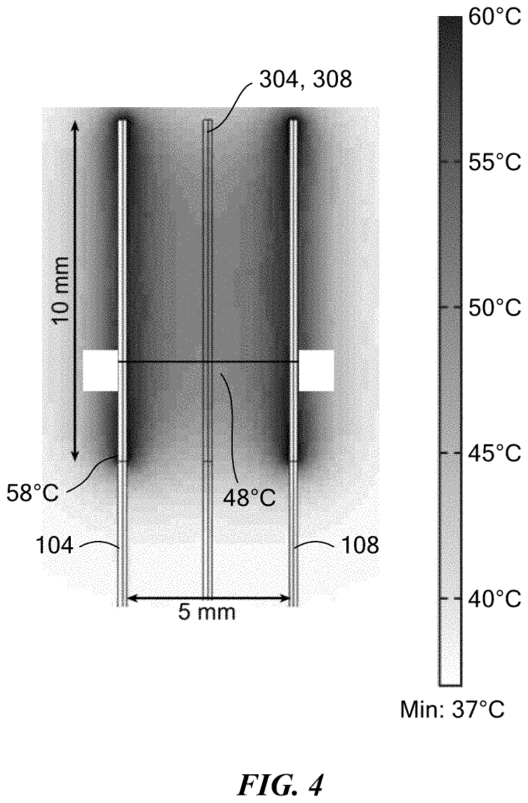

Two RF electrode needle pairs defining perpendicular planes was simulated and the thermal profile result is shown in FIG. 4. The simulation conditions were the same as the ones used for the single pair shown in FIG. 2. The difference of potential was 60 V applied for 20 sec, the needles were inserted in subcutis, and the active needle lengths were 10 mm. The thermal profile obtained with the two perpendicular pairs of RF energy delivering electrode needles was much more uniform than the one obtained with a single pair. The maximum temperature was still about 58° C. after 20 sec but the temperature of the midpoint between the two RF energy delivering electrode needles of both pairs was much higher: about 48° C. for the two-pair configuration instead of about 43° C. for the single pair configuration. As a result, using non-coplanar RF electrode needle pairs creates large thermal zones in tissue, such as the subcutis, and increases the uniformity of the thermal profile within the thermal volume.

Energy delivering needle electrodes 104, 108, 304 and 308, FIG. 3 are preferably parallel and preferably equidistant from intersection line 316 formed by intersection of the first and second planes. In one embodiment, the first plane 110 and the second plane 310 intersect at an angle of 15 to 90 degrees (or any other angle). In the particular case shown in FIG. 3, the angle between the first plane and the second plane is 90 degrees.

A supply of RF energy to needle electrodes 304 and 308 induces a current between electrodes 304 and 308 schematically shown by current streamlines 312. As noted above the angle between the first plane and the second plane is 90 degrees and current 312 flows in the direction perpendicular to current streamline 112 flowing between the first pair of energy delivering needle electrodes 104 and 108. The RF energy at intersection line 316, or within central volume 320, shown by streamlines 112 between energy delivering needle electrodes 104, 108 of the first pair, intersect the RF induced current shown by streamlines 312 between energy delivering needle electrodes 304, 308 of the second pair, to become a sum of the RF energies.

Accordingly, two or more pairs of needle electrodes are spaced about a larger area treatment zone and there is an energy subsystem for the needle electrodes such as RF generator 32a for needle pair 104, 108 and RF generator 32b for needle pair 304, 308. This ensures current flows only between needles 104 and 108 (generated by RF generator 32a) and only between needles 304 and 308 (generated by RF generator 32b). These RF generators may be electrically isolated via a transformer or they may be independently battery powered generators. There are, however, other means known in the art for electrically isolating the pairs of needles from each other and for inducing current from the first needle electrode of each pair to only the second needle electrode of the pair across the central volume of the zone.

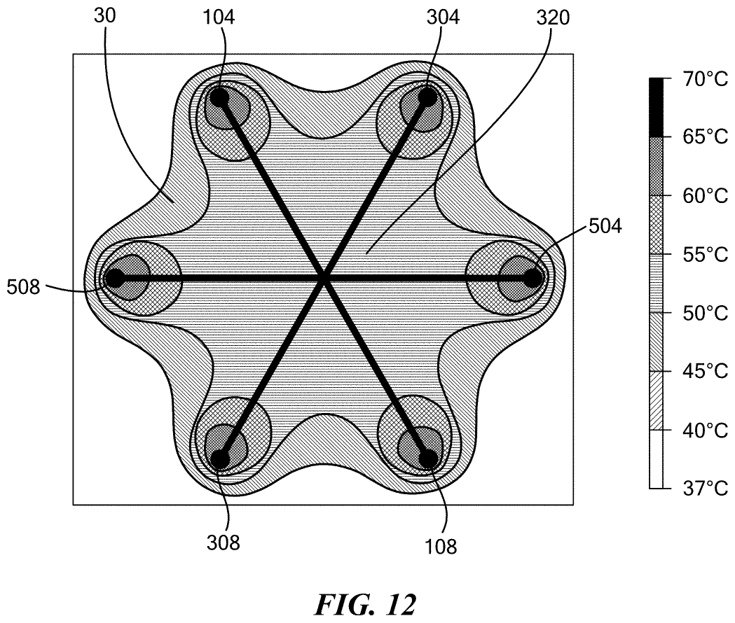

The RF generators are configured for supplying energy to each of the pairs of RF electrodes 104, 108, 304 and 308 in a controlled manner. Each pair of RF energy delivering needle electrodes is electrically insulated and separated from the other pair of RF energy delivering needle electrodes and each RF electrode pair could be independently and selectively activated, for example, by electrically isolating RF generators 32a, 32b feeding the pairs of electrodes from each other. See published U. S. Patent Application No. 2012/0143178 FIG. 12 and [0208]-[0210] incorporated herein by this reference. It would be also possible to only use one RF generator which would supply RF energy to one pair of electrodes at a time and then sequentially switching to other pairs during the treatment procedure. Typically, the switching time between the electrode pairs would be shorter than thermal relaxation time of the treated tissue. Thus, the energy source for each needle pair need not be a separate energy source for each needle pair.

Controller 34 may include a Personal Computer (PC). The PC may include a processor, one or more memories, a keyboard, a pointing device (mouse), and a display. The PC supports monitoring of the treatment process, changing the input parameters, and provides a graphical representation of an image of a target skin treatment volume. One or more programs stored in memory are executed by the processor to carry out the functionality described herein.

In this way, current is induced only between the needle electrode pairs 104, 108 and 304, 308 through the interior of the treatment volume 30 to increase the current density and temperature in the interior of the zone. In the specific embodiment of FIG. 3, the central volume 320 of the treatment zone 30 is where the current flows intersect but this is not a necessary limitation of the invention.

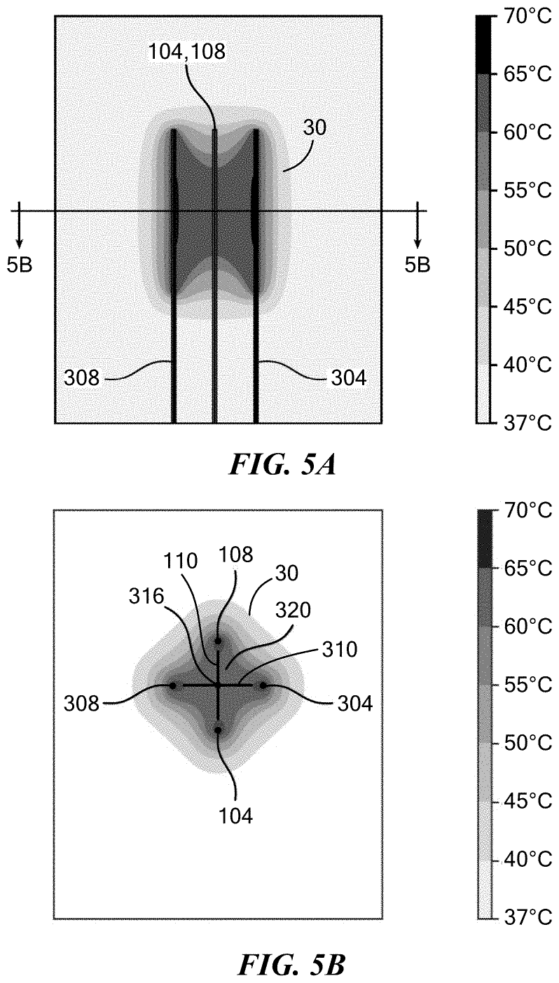

The uniformity of the thermal profile can be further improved by increasing the RF application time in order to allow more time for the heat front to propagate toward the central volume of the needle assembly. FIGS. 5A-5B show an example of a thermal profile of the same two pairs of long RF energy delivering needle electrodes arranged according to FIG. 3 where the RF application time was increased from 20 to 60 sec. The temperature of the interior 320 of treatment volume 30 is higher than the temperature caused by one pair of RF electrodes. The interior of treatment volume may be between 8 to 12 cm³. All energy delivering needle electrodes may be equidistant from axis 316 defined by the intersection of the two planes containing the individual electrode pairs.

FIGS. 5A and 5B also show that the thermal profile of such non-coplanar multiple electrically isolated RF needle pairs is contained along the active portion of the needles and between all needles creating the array. In these figures, two electrically isolated and independent pairs of needles were simulated. The distance between two needles of a pair was 5 mm, and a difference of potential of 60 V was applied between two needles of a common pair for 60 sec. The active length of each needles was 1 cm. The simulation was performed with the needles embedded in a medium having the same electrical and thermal properties as fat. The temperature within the treatment volume defined by the active portions of the needles is very uniform with a steep thermal gradient outside of the defined volume. This thermal characteristic is close to a perfect theoretical energy deposition in biological tissue or tissue ablation tool where, in theory, the thermal profile would create a step function where therapeutic temperature would be reached within a volume and non-therapeutic or normal body temperature outside the volume without any transition in between.

To quantify the temperature uniformity within the volume defined by the active parts of the needles of the array, a temperature smoothness ratio (SR) can be defined as:

SR=(Tₘₐₓ−Tᵢₙᵢₜ)/(Tₘᵢₙ−Tᵢₙᵢₜ)=ΔTₘₐₓ/ΔTₘᵢₙ, (1)

where:
Tₘₐₓ is the maximal temperature,
Tₘᵢₙ is the minimal temperature, and
Tᵢₙᵢₜ is the initial temperature.

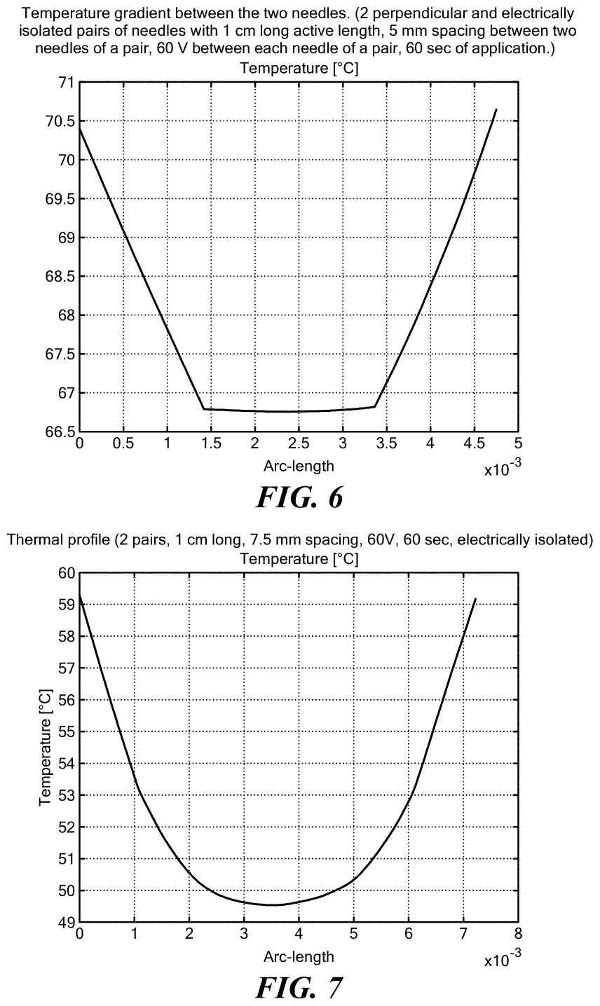

From the results discussed above, a calculated smoothness ratio of 1.12 indicated a very smooth thermal profile as shown in FIG. 6.

Increasing the inter electrode distance to 7.5 mm seemed to be a maximal limit in order to maintain a smooth thermal profile. Indeed, the simulation results shown in FIG. 7 show that the calculated smoothness profile increased from 1.12 to 1.76 therefore showing a greater thermal gradient across the needles, which is less desirable than a smoother thermal gradient across the needles. An inter electrode distance beyond about 7.5 mm has therefore a negative effect on the thermal profile smoothness.

The embodiments presented so far include of applying a fix voltage between the two needles of a common pair for a fixed amount of time. Instead of applying a fix electrical parameter such as a voltage, a current, or a power, a better way would be to use a temperature-control algorithm to precisely control the temperature within the treated area. To do so, a temperature sensor (such as a thermocouple) can be positioned within at least one needle of the arrangement shown in FIG. 3 for example, more preferably within at least one needle of each independent pair. In a preferred embodiment associated with the needle arrangement example of FIG. 3, one temperature sensor would be located within the needle 108 or 104, and another temperature sensor would be located within the needle 304 or 308. The temperature sensor within needle 104 or 108 would be used by the controller 34 to control the electrical power delivered by the RF generator 32a to reach and maintain a pre-defined target temperature 301. Similarly, the temperature sensor within needle 304 or 308 would be used by the controller 34 to control the electrical power delivered by the RF generator 32b to reach and maintain a pre-defined target temperature 301.

Along with the temperature sensor and as described above, a controller is used to receive the in situ tissue temperature information and control the electrical power delivered to the needle pair to reach and maintain a pre-determined tissue temperature. Examples of commonly used controllers to reach and maintain a target, a temperature in this case, are PID (Proportional-Integral-Derivative) or PI (Proportional-Integral) controllers. Other types of controllers can also be used to reach and maintain a target temperature. In the case of electrically isolated electrode pairs described in FIG. 3, one RF generator is used to deliver the electrical power to one pair of electrode needle. Each independent RF generator can therefore be controlled by an independent controller, such as a PID or a PI controller—each of these controllers receiving inputs from the temperature sensors of their associated electrode needle pairs to reach and maintain the target temperature 301 selected by the user. The target temperature associated to one needle pair is reached when the temperature sensor located within at least one electrode of the pair is reached. Using the same target temperature 301 for all the needle pairs in the configuration described in FIG. 3 has for effect to create a smooth thermal profile within the zone 30. Since the controllers can control the generators to reach and maintain a precise tissue target temperature for a specified amount of time within a volume defined by the active parts of the needles, a time-temperature dose known to produce desirable biological effects can be delivered within the volume 30. This configuration could be used to deliver a precise dose of thermal energy to remove produce an apoptotic and/or necrotic response in fat for example, for procedures aimed at reducing the amount of fat in a body area such as the submental space, the thighs, or the abdomen for example. Although several controllers have been described, one skilled in the art could appreciate that a single controller could also be used to control all RF generators by sequentially controlling all generators independently one after another. The main concept expressed in this section is section is to use tissue temperature feedback and control each pair of needles to reach and maintain a pre-defined tissue temperature.

When a temperature-control algorithm is employed, a target tissue temperature is selected by the user, usually from a GUI (Graphical User Interface). The target temperature could also have a fixed value which is pre-programmed within the software or hardware controlling the RF generator(s). For example, a temperature input 301 is selected by the user, which is used as a target temperature value by the controller(s).

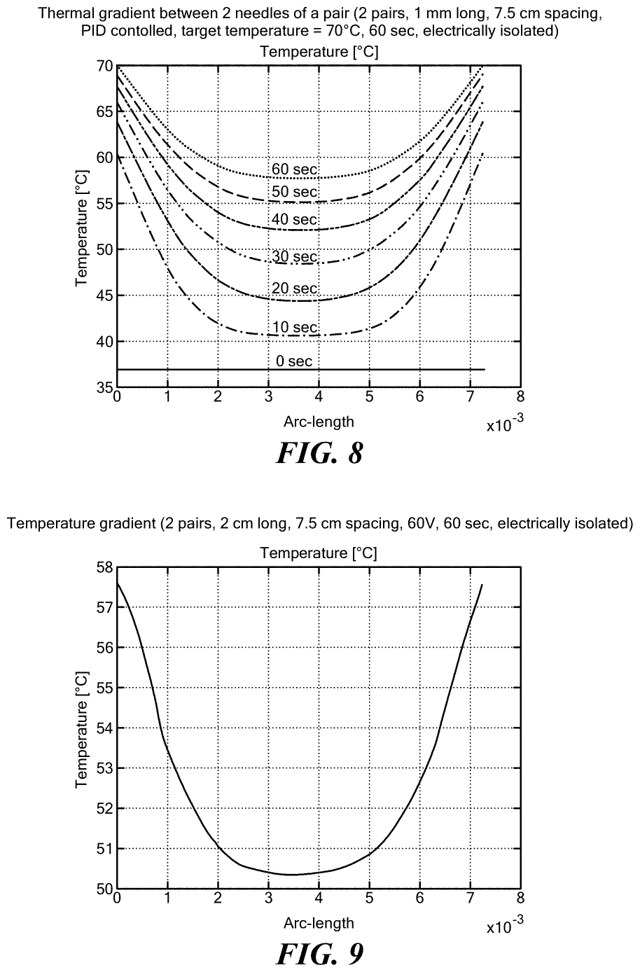

Controller 34, FIG. 3 may be programmed to control RF generators 32a and 32b based on the temperature signal provided to the controller from a temperature sensor such as one or more thermocouples. The thermal profile smoothness can be improved as shown in FIG. 8, which is a simulation of the needle arrangement with a temperature feedback point along the active length of the needle, and using a PI controller where the target temperature was set at 70° C. The smoothness factor is 1.61 after 60 seconds of application. The target temperature of 70 degrees C. was reached after 60 seconds.

As stated earlier, tissue temperature control methods may include a temperature sensor, like a thermocouple or a thermistor for example, within at least one needle of a pair, and a controller, such as or including a PID (Proportional-Integral-Derivative) or a PI (Proportional-Integral) controller, for example, in order to reach and maintain a pre-determined tissue temperature. The optimal location of the temperature sensor within the needle is anywhere along the active portion and preferably where the hottest temperature spot is located. The controller monitors the tissue temperature by reading the temperature of the temperature sensor located within at least a needle at a pre-determined sampling frequency, such as 10 Hz for example, and adjust the difference of potential between two needles of a pair to reach and maintain a predefined tissue temperature target. In similar embodiments, the controller could control the power applied in tissue by the needle pair or the current between the needles of a pair.

Using such a technique, along with the capability of the disclosed needle configurations, creates a fairly uniform thermal pattern within the treated volume allowing a user to deliver RF energy to create precise target tissue temperature and/or a precise thermal dose which has been clinically proven to achieve the therapeutic goals. For example, the time-temperature profile can be characterized to create irreversible damage to fat cells, or adipocytes, to bring them out of their viable range. See Weaver, J. A., and Stoll, A. M., Mathematical Model of Skin Exposed to Thermal Radiation, Aerospace Medicine, January 1969, pp 24-30, and Weaver, J. A., and Stoll, A. M. Mathematical Model of Skin Exposed to Thermal Radiation, Aerospace Medical Research Department, Department of the Navy, AirTask R01 101 01 (Task Problem No. RB-6-01) (Aug. 22, 1967), both incorporated herein by this reference. More specifically, irreversible damages were created when adipocyte temperature of 45° C. was maintained for 40 minutes. Since biological tissues are sensitive thermal dose, similar biological effects can be obtained at higher temperature and lower time, or lower temperature and higher time. Equivalent thermal effect can be determined using the Arrhenius equation, which is known by those skilled in the art. According to the data from the above cited papers, similar irreversible damages to adipocytes could be induced at temperatures of 46, 47, and 48° C., for durations of 16, 6, and 3 minutes, respectively. These settings could be useful when fat reduction is the objective of a medical or esthetic procedures, and when temperature feedback algorithm is used to reach and maintain a target temperature.

The example above is one treatment example only and many other treatment examples associated with subcutis and/or other biological tissues are possible.

There may be a limit to the inter electrode distance where a uniform thermal profile can be expected. With an inter electrode distance of 1 cm, the middle section temperature did not rise substantially and the smoothness factor was 3.33. Longer needles (2 cm active length) were then simulated with satisfactory results and FIG. 9 shows a temperature profile between the two longer needles where the calculated smoothness ratio was 1.52. Simulations with even longer needles, up to 40 mm, were also performed and all underlying principles described herein were valid. Therefore, the active needle length can be as short as 0.5 mm, or 3 mm or up to 40 mm and above.

The RF energy delivering needle electrodes can be spaced apart from each other and from the central volume intersection axis by 1.0 mm to 10 mm. Simulations have shown that the most optimal inter-electrode distance to minimize the SR value defined above for the same pair is 7 to 8 mm. Usually, the RF energy delivering needle electrodes are made of the same length although different length electrode pairs could be used in some applications.

Subcutis (fat) was selected for the simulations because it arguably has the lowest electrical (σ) and thermal (k) conductivities of all biological tissues and presents a situation where it is very difficult to obtain large and uniform thermal profiles. Nonetheless, treatments of other biological tissues are possible using the new techniques described herein.

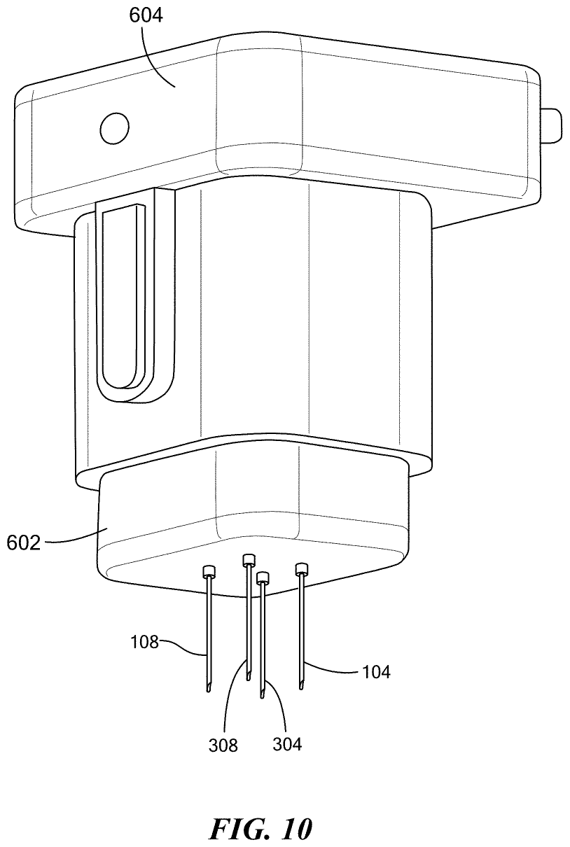

The pairs of needle electrodes may be mounted on a detachable and replaceable cartridge 602, FIG. 10. Cartridge 602 can be removably attached to a handle 604 or an applicator (see FIG. 1) such that the RF needle electrodes extend distally from the handle or applicator. The distal tip of each RF energy delivering needle electrode is preferably sharp such that it is capable of piercing tissue. The cartridge may be spring loaded to deploy the needles out of the cartridge when the cartridge is pressed onto the skin. The needle end proximal to cartridge 602 of the RF energy delivering needle electrodes can be insulated. Insulation along a segment of the lengths of RF electrodes may reduce undesirable delivery of RF energy to non-target tissues. Such insulated electrodes comprise lengths sufficient to penetrate/position the non-insulated portions of the RF energy delivering needle electrodes to a desired depth. The insulated segments of energy delivering needle electrodes can be made of electrically non-conductive materials or materials with a low electrical conductivity when compared to biological tissue. Suitable electrically non-conductive materials include such as plastic, silicone, Teflon, ceramic, or the like.

The RF energy delivering needle electrodes 104, 108, 304, and 308 could be made of regular hypodermic needles with gauge 12 (2.769 mm) to 34 (0.16 mm). A hypodermic needle has sharp ends to easily penetrate the skin and can be advanced into the skin/tissue the whole lengths (about 40 mm) of the needle. The hypodermic needle is also a hollow tube that could allow for a supply of fluids to the treatment volume. The preferred needles are made of medical grade steel although needles made of stainless steel, platinum, gold or silver could be used. The hypodermic needles are preferably sufficiently rigid to maintain the distance between them constant when inserted in the tissue. The hypodermic needles and non-conductive insulation layer are also sufficiently robust to sustain multiple penetrations into and retraction from biological tissue during typical treatment procedures.

The multiple non-coplanar needle pairs configurations described so far are arranged in a spatial configuration to work together in order to create a large and uniform thermal profile in biological tissue. This needle array arrangement could be considered as a unit cell. It is also possible to use a plurality of unit cells to increase the treatment volume by simultaneously inserting these unit cells in tissue. If the unit cells are spaced apart closely enough, the corresponding thermal patterns would be uniform from one unit cell to another. Conversely, the thermal patterns would show a zone of untreated tissue where the temperature levels are below a therapeutic threshold between the unit cells. Depending on the treatment requirements, the distance between unit cells could be varied to produce large and uniform thermal injuries or to leave zones of untreated tissue between the unit cells.

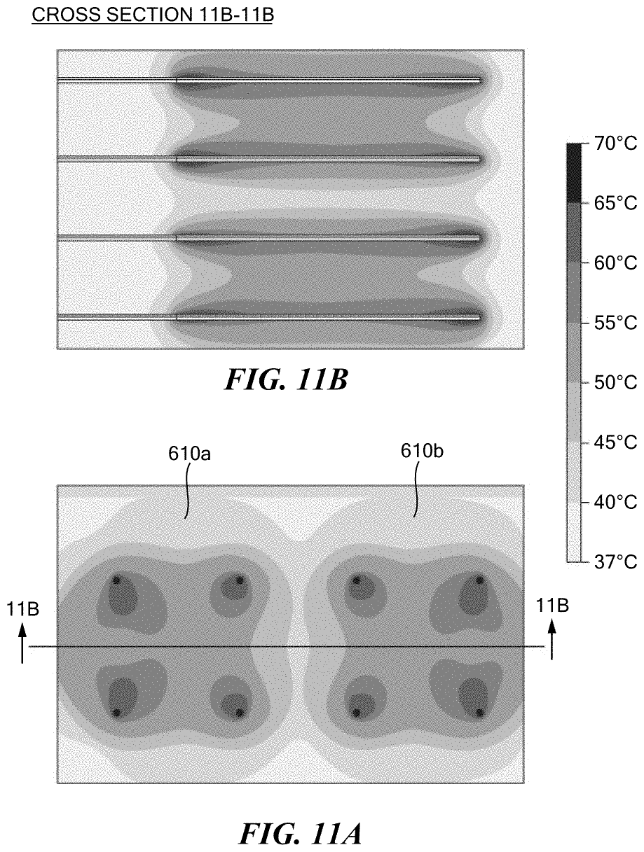

FIGS. 11A-11B show an example of two unit cells, 610a, 610b each having two pairs of RF energy delivering needle electrodes. This configuration therefore includes four pairs of RF energy delivering needle electrodes. In this case, the active length of the needles was 40 mm, the distance between two needles of a common pair was 7.5 mm, and the distance between the two unit cells was 5 mm. A potential of 60 V was applied between the two needles of each pair and the RF energy was applied for 60 seconds. In this specific example, the simulation resulted in these values:
Electrical impedance of each needle electrode pair: 4800Ω
RF Power per electrode pair: 0.75 W
RF Energy per electrode pair: 45 J
Current per electrode pair: 12.5 mA

The frequency was 460 kHz which is normally used for cardiac and cancer RF ablation. It is worthwhile noting that higher frequencies, up to 5 MHz for example, could also be used.

Although a square RF electrode cell configuration has been shown, other RF energy delivering needle electrode configurations are possible such as hexagonal, octagonal, and the like. FIG. 12 is an example of an RF energy delivering needle electrode configuration with three needle pairs 104, 108; 304, 308; and 504, 508.

The device disclosed is suitable to treat cellulite, circumference reduction of the abdomen, and body shaping, including fat reduction in the submental and neck area. The device can be used to deliver thermal energy to a large tissue mass such as cancer, uterine fibroid, and the like. Because of the small footprint of the needles, the large thermal profile, and the controllability of the thermal profile which is limited along the active length of the needles and within the cells defined by the needle configuration, the device would be beneficial for brain tumors and other uses where the protection of surrounding healthy tissue is of primary importance.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments.

In addition, any amendment presented during the prosecution of the patent application for this patent is not a disclaimer of any claim element presented in the application as filed: those skilled in the art cannot reasonably be expected to draft a claim is that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant cannot be expected to describe certain insubstantial substitutes for any claim element amended.

Other embodiments will occur to those skilled in the art and are within the following claims.

What is claimed is:

1. A device for treating tissue, the device comprising:
two or more pairs of needle electrodes spaced about a zone;
each pair of needle electrodes including a first needle electrode on one side of the zone and a second needle electrode on an opposite side of the zone; and
an energy subsystem configured to induce current from the first needle electrode of the pair to only the second needle electrode of the pair through the interior of the zone to increase the total current density and temperature in the interior of the zone.

2. The device of claim 1 in which each pair of needle electrodes defines a plane intersecting the center of the zone.

3. The device of claim 2 in which the planes defined by each pair of needle electrodes intersect each other at the center of the zone.

4. The device of claim 1 in which the energy subsystem includes an RF generator for each needle electrode pair connected to the first and second needle electrodes of each pair.

5. The device of claim 4 in which the RF generator of each pair of needle electrodes is electrically isolated from the RF generators for all other pairs of needle electrodes.

6. The device of claim 1 in which the needle electrodes are all equidistantly spaced from the center of the zone.

7. The device of claim 1 further including a cartridge carrying the pairs of needle electrodes.

8. The device of claim 7 further including an applicator for receiving the cartridge.

9. The device of claim 8 in which the cartridge is removeable from and insertable into the applicator.

10. The device of claim 1 in which each needle electrode has an active length of between 0.5 and 40 mm.

11. The device of claim 1 in which the needle electrodes of each pair of needle electrodes are spaced apart from each other by a distance of between 1.0 and 10 mm.

12. The device of claim 11 in which each needle electrode is spaced apart from each adjacent needle electrode by a distance of between 0.71 and 7.1 mm.

13. The device of claim 1 in which one or more needle electrodes further includes a temperature sensor.

14. The device of claim 13 further including a controller for said energy subsystem responsive to said one or more temperature sensors.

15. The device of claim 14 in which the controller is configured to control the energy subsystem based on an output signal of said one or more temperature sensors.

16. The device of claim 15 in which the controller is configured to control the energy subsystem to reach and maintain a predetermined temperature in the zone.

17. The device of claim 16 in which the controller is configured to automatically adjust the temperature in the zone to between 40° C. and 48° C. from between 30 seconds and 30 minutes to irreversibly damage adipocytes in the zone.

18. A method of treating tissue, the method comprising:
inserting two or more pairs of needle electrodes into a zone of tissue, each pair of needle electrodes including a first needle on one side of the zone and a second needle on an opposite side of the zone;
electrically isolating each pair of needle electrodes from all other pairs of needle electrodes; and
inducing current from the first needle electrode of each pair to only the second needle electrode of the pair through the interior of the zone increasing the total current density and temperature at the interior of the zone.

19. The method of claim 18 in which each pair of needle electrodes define a plane intersecting the center of the zone.

20. The method of claim 19 in which the planes defined by each pair of needle electrodes intersect each other at the center of the zone.

21. The method of claim 18 in which the needle electrodes are all equidistantly spaced from the center of the zone.

22. The method of claim 18 in which each needle electrode has an active length of between 0.5 and 40 mm.

23. The method of claim 18 in which the needle electrodes of each pair of needle electrodes are spaced apart from each other by a distance of between 1.0 and 10 mm.

24. The method of claim 23 in which each needle electrode is spaced apart from each adjacent needle by a distance of between 0.71 and 7.1 mm.

25. The method of claim 18 further including sensing the temperature proximate one or more needle electrodes.

26. The method of claim 25 further including controlling the current induced between the needle electrodes based on the temperature sensed proximate said one or more needle electrodes.

27. The method of claim 26 including controlling the current induced between the needle electrodes to reach and maintain a predetermined temperature in the zone.

28. The method of claim 27 including automatically adjusting the temperature in the zone to between 40° C. and 45° C. from between 30 seconds and 30 minutes to irreversibly damage adipocytes in the zone.

29. A device for treating tissue, the device comprising:
pairs of needle electrodes electrically isolated from each other and spaced about a zone having a central volume;
each pair of needle electrodes defining a plane intersecting the central volume of the zone;
the planes defined by each pair of needle electrodes intersecting each other at the central volume of the zone; and
means for inducing a current from the first needle electrode of each pair to only the second needle electrode of each pair across the central volume of the zone.

30. A method of treating tissue, the method comprising:
inserting a plurality of needles into a tissue treatment zone;
spacing the needles from a central volume of the tissue treatment zone;
inducing current from each needle on one side of the central volume of the treatment zone through the central volume of the treatment zone to only another needle on an opposite side of the central volume of the treatment zone; and
controlling the induced currents to intersect in the central volume of the treatment zone increasing the energy deposition in the central volume of the treatment zone.

31. The method of claim 30 in which the plurality of needles are spaced equidistant from the central volume of the treatment zone.

32. A device for treating tissue, the device comprising:
two or more pairs of needle electrodes spaced about a zone;
each pair of needle electrodes including a first needle electrode on one side of the zone and a second needle electrode on an opposite side of the zone;
an energy subsystem configured to induce current from the first needle electrode of the pair to only the second needle electrode of the pair through the interior of the zone to increase the total current density and temperature in the interior of the zone;
at least one temperature sensor for measuring a temperature in the zone; and
a controller for the energy subsystem responsive to the temperature sensor and configured to adjust the energy subsystem to reach and maintain a predetermined temperature in the zone.

33. The device of claim 32 in which the energy subsystem is an RF generator for each pair of needle electrode pairs connected to the first and second needle electrodes of each pair.

34. The device of claim 32 in which the controller is configured to automatically adjust the temperature in the zone to between 40° C. and 48° C. from between 30 seconds and 30 minutes to irreversibly damage adipocytes in the zone.

35. A method of treating tissue, the method comprising:
providing two or more pairs of needle electrodes for insertion into a zone of tissue, each pair of needle electrodes configured to include a first needle to be located on one side of the zone and a second needle to be located on an opposite side of the zone;
electrically isolating each pair of needle electrodes from all other pairs of needle electrodes;
inducing current from the first needle electrode of each pair to only the second needle electrode of the pair through the interior of the zone increasing the total current density and temperature at the interior of the zone;
sensing a temperature in the zone; and
adjusting the current induced from the first needle electrode of each pair to the second needle electrode of each pair to reach and maintain a predetermined temperature in the zone.

36. The method of claim 35 in which adjusting the current includes automatically adjusting the temperature in the zone to between 40° C. and 48° C. from between 30 seconds and 30 minutes to irreversible damage adipocytes in the zone.

37. A method of treating tissue, the method comprising:
inserting two or more pairs of needle electrodes into a zone of tissue, each pair of needle electrodes including a first needle on one side of the zone and a second needle on an opposite side of the zone;
selecting a temperature to be reached and maintained in the zone; and
inducing current from the first needle electrode of each pair to only the second needle electrode of the pair through the interior of the zone to reach and maintain said selected temperature in the zone.

38. The method of claim 37 in which the temperature selected is between 40° C. and 48° C.

39. The method of claim 37 further including maintaining said temperature for a selected period of time.

40. The method of claim 39 in which said selected time period is between 30 seconds and 30 minutes.

41. A cartridge for a tissue treatment device, the cartridge comprising:
two or more pairs of needle electrodes to be spaced about a zone;
each pair of needle electrodes including a first needle electrode to be located on one side of the zone and a second needle electrode to be located on an opposite side of the zone; and
each paid of needle electrodes configured so a current is induced from the first needle electrode of the pair to only the second needle electrode of the pair through the interior of the zone in order to increase the total current density and temperature in the interior of the zone.

42. The cartridge of claim 41 in which each pair of needle electrodes defines a plane intersecting the center of the zone.

43. The cartridge of claim 42 in which the planes defined by each pair of needle electrodes intersect each other at the center of the zone.

44. The cartridge of claim 41 in which the needle electrodes are all equidistantly spaced from the center of the zone.

45. The cartridge of claim 41 in which the cartridge is removeable from and insertable into an applicator.

46. The cartridge of claim 41 in which each needle electrode has an active length of between 0.5 and 40 mm.

47. The cartridge of claim 41 in which the needle electrodes of each pair of needle electrodes are spaced apart from each other by a distance of between 1.0 and 10 mm.

48. The cartridge of claim 41 in which each needle electrode is spaced apart from each adjacent needle electrode by a distance of between 0.71 and 7.1 mm.

49. A cartridge for treating tissue, the cartridge comprising:
pairs of needle electrodes electrically isolated from each other and spaced about a zone having a central volume;
each pair of needle electrodes defining a plane intersecting the central volume of the zone;
the planes defined by each pair of needle electrodes intersecting each other at the central volume of the zone; and
the pairs of needle electrodes configured so a current is induced from the first needle electrode of each pair to only the second needle electrode of each pair across the central volume of the zone.
